(19) United States
(12) Patent Application Publication
(10) Pub. No.: US 2011/0117173 A1
(43) Pub. Date: May 19, 2011

(54) POWDERED CHEWING GUM COMPOSITIONS, THE USE THEREOF AND A METHOD OF PREPARING SUCH COMPOSITIONS

(75) Inventors: Jens Hummeluhr, Charlottenlund (DK); Otto Andresen, Stenlose (DK); Tage Vedsted Kusk, Klippinge (DK)

(73) Assignee: ALSIANO A/S, Birkerød (DK)

(21) Appl. No.: 13/002,657

(22) PCT Filed: Jul. 7, 2009
(86) PCT No.: PCT/DK2009/050166

§ 371 Date: Feb. 3, 2011

(30) Foreign Application Priority Data
Jul. 8, 2008 (EP) 08012295.5

(51) Int. Cl. A23G 4/20; A61K 9/68; A61K 35/60; A61K 31/20; A61K 35/74; A61K 31/522; A61K 36/77; A61K 38/43; A23G 4/06

(52) U.S. Cl. 424/440; 424/523; 514/560; 424/93.4; 514/263.34; 424/725; 424/94.1; 426/5

(57) ABSTRACT

The present invention relates to a powdered chewing gum composition, which contains over 50% w/w chewing gum base and wherein at least 95% w/w of the particles of the composition have a particle size of less than 1200 micron in diameter. The present invention also relates to the use of said powdered chewing gum composition. The present invention also relates to a chewing gum comprising a) a core component, and b) one or more layers comprising the composition of the present invention. The present invention further relates to a method for production of said chewing gum comprising the steps of: (a) providing a core composition; and (b) coating of the core composition with a powdered chewing gum composition by use of a panning process; and optionally (c) coating the outer intermediate layer with an outer finishing layer by use of a panning process.

### TECHNICAL FIELD

The present invention relates to powdered chewing gum compositions, the use of such compositions, and a method for preparing such compositions.

### BACKGROUND OF THE INVENTION

Production of chewing gum has traditionally been carried out using expensive and dedicated production equipment, which due to the nature of the processes employed, requires large investment. It has therefore hitherto only been profitable to produce chewing gum in large production units. As a result, there are only few chewing gum producers in the world.

In the later years, it has become possible to produce chewing gum tablets by compressing of powdered chewing gum compositions. This is carried out in tabletting machines, which is standard equipment in the pharmaceutical industry, but normally not available to the confectionery industry. Accordingly, there is a need for an alternative production method for producing chewing gum from powdered chewing gum compositions that can be carried out with equipment widely available in small as well as large companies in the confectionery industry, and not requiring heavy investment in expensive apparatus.

Such a method has been described briefly in EP 0814 670 B1. The method uses conventional panning equipment, which is widespread in the confectionery industry, as well as in the pharmaceutical industry. The applicants have, however, observed that said method has a low production yield when carried out with commonly available powdered chewing gum compositions. The low yield is caused by poor adherence of the powdered chewing gum composition particles to the centres being coated. Instead, the powdered chewing gum composition accumulates on the walls of the panning apparatus.

U.S. Pat. No. 6,551,634 B2 discloses chewing or bubble gum pieces comprising a flavouring containing core, an intermediate gummy mass free layer which encapsulates the core, and an outer layer which is an outer layer of a gum piece that is placed in the mouth of a person chewing the gum and contains gummy mass, which outer layer encapsulates the intermediate layer. The outer gummy mass containing layer is applied as a pulverised material. The chewing or bubble gum pieces may comprise a core, which instead of or as a supplement to the flavourings contains medicine mass.

In U.S. Pat. No. 6,558,722 B2, a powdered chewing gum composition is applied containing between about 5 and about 40% chewing gum base. It is mentioned that “if too much dry charge is applied to the wetted cores, the overload of dry charge collects in the back of the pan.” It is never specified in a quantitative way what “too much” means. It is therefore implicitly suggested that it is problematic to carry out the application of the dry charge with said powdered chewing gum composition.

European Patent Application No. 1350434 relates to a chewing gum in powder form comprising gum base, referred to as “soft basic gum”, together with a sweetener and other typical chewing gum ingredients. The obtained powder can be compressed to form a chewing gum. Within the powdered composition, gum base can have a concentration of up to 48.5% by weight.

U.S. Pat. No. 3,152,908 discloses a method for manufacturing a glazed chocolate product in a panning process. The dry glaze composition contains 70% gum arabic together with other water-soluble ingredients, the composition being not suitable for forming a water-insoluble gum base of a chewing gum coating.

U.S. Pat. No. 4,456,629 relates to a method for forming a shiny wax coating allowing for printing over the shiny wax coated tablet. There is no indication as to whether or how this technology might be applicable or relevant to chewing gum coatings.

International Patent Application WO 99/13870 teaches a method for producing a chewing gum, wherein the chewing gum composition comprises the physiological cooling agent acyclic carboxamide. Within the composition, the cooling agent is preferably encapsulated by an encapsulating agent comprising maltodextrin or gum arabic. For manufacturing chewing gum, the various ingredients are mixed, discharged from the mixer and shaped into the desired form by rolling and cutting, extrusion or casting. There is no pointer towards coating of centres with chewing gum, or how the disclosed composition might be adapted to that end.

Similarly, U.S. Pat. No. 4,238,475 relates to a chewing gum composition comprising therapeutic components, the latter being coated with gum arabic. The chewing gum contains gum base in the range of 8 to 25% by weight and is produced by mixing the various ingredients of the composition. There is made no mention of coating of centres with chewing gum composition, or how the disclosed mixture would be useful or suitable in this respect.

Accordingly, there is still a need in the art for alternative powdered chewing gum compositions that are suitable for production of chewing gum by panning.

Accordingly, an object of the present invention is to provide alternative powdered chewing gum compositions and alternative means of producing chewing gum, and to provide use of the powdered chewing gum compositions for producing chewing gum.

### SUMMARY OF THE INVENTION

The applicants have now surprisingly observed that by increasing the content of chewing gum base in a powdered chewing gum composition to more than 50% w/w, said poor adherence of the powdered chewing gum composition particles to the centres being coated in a standard panning process is overcome.

Thus, the present invention relates to a powdered chewing gum composition which can be applied for coating of any edible and chewable centres in a repeated panning process. The powdered chewing gum composition contains over 50% w/w of chewing gum base, preferably more than 65% w/w of chewing gum base, more preferably more than 75% w/w of chewing gum base. At least 95% w/w of the particles of the composition have a particle size of less than 1200 micron in diameter.

The chewing gum base used in the powdered chewing gum composition of the present invention may have a softening point between 50° C. and 65° C., preferably 60° C.

In a preferred embodiment, the powdered chewing gum composition according to the present invention is a powder which is free flowing at temperatures between 5° C. and 30° C.

Preferably, the powdered chewing gum composition according to the invention contains up to 100% but at least 50% w/w of chewing gum base, such as at least 51% w/w, such as at least 52%, at least 53%, at least 54%, at least 55% w/w, such as at least 56%, at least 57%, at least 58%, or at least 59% w/w of chewing gum base.

Even more preferably, the powdered chewing gum composition according to the invention contains up to 100% but at least 60% w/w of chewing gum base, such as at least 61% w/w, such as at least 62%, at least 63%, at least 64%, at least 65% w/w, such as at least 66%, at least 67%, at least 68%, or at least 69% w/w of chewing gum base.

Even more preferably, the powdered chewing gum composition according to the invention contains up to 100% but at least 70% w/w of chewing gum base, such as at least 71% w/w, such as at least 72%, at least 73%, at least 74%, at least 75% w/w, such as at least 76%, at least 77%, at least 78%, or at least 79% w/w of chewing gum base.

Even more preferably, the powdered chewing gum composition according to the invention contains up to 100% but at least 80% w/w of chewing gum base, such as at least 81% w/w, such as at least 82%, at least 83%, at least 84%, at least 85% w/w, such as at least 86%, at least 87%, at least 88%, or at least 89% w/w of chewing gum base.

Said powdered chewing gum composition may contain other ingredients well known in the art, such as flow-improving agents, antioxidants, colourants, flavours, sugars or sugar replacers, e.g. bulk sweeteners, polyols, high intensity sweeteners. As examples of flow-improving agents can be mentioned talc, silica, magnesium stearate, gum arabic and the like. Well known antioxidants include BHT, tocopherol, sodium erythorbate etc.

In order to accommodate additional ingredients, the powdered chewing gum composition according to the invention in a preferred embodiment contains less than 100% w/w of chewing gum base, such as less than 99% w/w, such as less than 98%, less than 97%, less than 96%, less than 95% w/w, such as less than 94%, less than 93%, less than 92%, or less than 91% w/w of chewing gum base.

In order to be applicable in the panning process, over 95% w/w of the particles of said free flowing powdered chewing gum composition must be below 1200 micron. Preferably, more than 65% of the particles are between 100 and 1000 micron. This has the advantage that the particle size distribution of the inventive composition is similar to the particle size distribution of potential additive powdered compositions such as flavouring agents or the like, thereby facilitating the mixing process.

### DEFINITIONS

The term “chewing gum” as used herein refers to a type of chewable confectionary, part of which is not dissolved during chewing.

The term “chewing gum base” as used herein refers to the part of a chewing gum which is not dissolved during chewing. Chewing gum compositions usually comprise a water soluble bulk portion, a water insoluble chewing gum base portion together with flavouring agents. During chewing, the water soluble portion dissipates with a portion of the flavouring agent. The chewing gum base portion is substantially retained in the mouth throughout the chewing process. Chewing gum base comprises hydrophobic, elastic and non-water soluble substances that enable it to be chewed for a long time without experiencing substantial changes. In other words, chewing gum base as used herein, is the fraction of chewing gum that is water-insoluble and non-digestible. Chewing gum base generally comprises elastomers, resins, fats and oils, waxes, softeners and inorganic fillers. Elastomers may include polyisobutylene, isobutylene-isoprene copolymer and styrene butadiene rubber, as well as natural latexes such as chicle. Resins include polyvinylacetate and terpene resins. Fats and oils may also be included in the chewing gum base, including tallow, hydrogenated and partially hydrogenated vegetable oils, and cocoa butter. Commonly employed waxes include paraffin, microcrystalline and natural waxes such as beeswax and carnauba. The chewing gum base typically also includes a filler component. The filler component may be calcium carbonate, magnesium carbonate, talc, dicalcium phosphate or the like. Chewing gum bases typically also contain softeners, including glycerol monostearate and glycerol triacetate. Further, chewing gum bases may also contain optional ingredients such as antioxidants, colours, and emulsifiers. The definition of chewing gum base as used herein, explicitly excludes gum arabic or similar substances, which are highly soluble in water.

The term “powdered chewing gum composition” as used herein refers to a powder-formed mixture of “chewing gum base” and sweeteners and flavours as well as functional ingredients which may comprise texture regulating agents, flow-improving agents, anticaking agents, antioxidants, colours or the like.

Texture regulating agents may be part of the powdered chewing gum composition in order to optimize the chewability and mouth feel of the chewing gum. Texture regulating agents, also known in the art as plasticizers or plasticizing agents, may include hardened fat, glycerine, lecithin and combinations thereof.

It is noted that in some technical fields, the term “gum” may not be used in the context of the insoluble part of a confectionary as described above, but rather may relate to water-soluble or water-dispersible carbohydrate based hydrocolloids. For example, Kirk-Othmer: Concise Encyclopedia of Chemical Technology, John Wiley & Sons, 1985, has the following definitions: “Commercial gums are water-soluble or water-dispersible hydrocolloids” (pp. 202-203). “The term gums denote a group of industrially useful polysaccharides or their derivatives that hydrate in hot or cold water to form viscous solutions or dispersions” (p. 572 ff). These gums include gum arabic (gum acacia), gum tragacanth, locust bean gum, guar gum, dextran, xanthan gum, agar-agar, carrageenan and several others. They all lack the most important property of chewing gum, to remain in the mouth even after prolonged chewing. Therefore, this terminology relating to “gums” as such is not applicable to the teachings presented herein.

The term “softening point” as used herein refers to a temperature measured by the Ring-and-Ball assay known in the art.

The term “free flowing” as used herein in relation to a powder means that said powder moves like a liquid when poured.

The term “flow-improving agent” as used herein means any substance which may be added to a powder in order to make it free flowing.

The term “standard panning apparatus” as used herein refers to apparatus for panning as e.g. the Driacoater® produced by Driam GmbH, various coating pans produced by A.M.P-Rose in the U.K., and others. A “panning process” is defined as a process for making e.g. chewing gum in which layers are slowly deposited onto a core by spinning the chewing gum in a horizontally-mounted pan also know as a “standard panning apparatus”. The process is well known to the person skilled in the art.

The term “cud-size” as used herein means the non-soluble part which remains after all other ingredients contained in the original chewing gum piece have been dissolved by chewing.

By w/w is meant the weight based on dry weight.

### DETAILED DESCRIPTION OF THE INVENTION

The applicants have surprisingly observed that by increasing the content of chewing gum base in a powdered chewing gum composition to more than 50% w/w, the aforementioned poor adherence of the powdered chewing gum composition particles to the centres being coated is avoided. Thus, the present invention relates to a powdered chewing gum composition, containing over 50% w/w chewing gum base wherein at least 95% w/w of the particles of the powdered chewing gum composition have a particle size of less than 1200 micron. A suitable gum base used in the composition of the present invention may, for example, correspond to the “soft basic gum” disclosed in EP 1350434 A1 on page, lines 30-34.

Best results are seen when the powdered chewing gum composition contains between 50% w/w and 90% w/w chewing gum base. Even better results are seen when the powdered chewing gum composition contains between 65% w/w and 85% w/w chewing gum base.

It is preferred that the chewing gum base used in the compositions according to the invention has a softening point of between 50-65° C., most preferably between 58-62° C., because a softening point lower than 58° C. makes the powder preparation sticky at the process temperature, reducing the desired free flowability. A softening point higher than 62° C. makes the final gum uncomfortably hard to chew. The interval of 50-65° C. appears to suitably delimit the acceptable range of softening points for chewing gum base used in the present invention.

According to a preferred embodiment, the chewing gum base used for the composition of the present invention comprises elastomers. Elastomers may include polyisobutylene, isobutylene-isoprene copolymer and styrene butadiene rubber, as well as natural latexes such as chicle.

According to another embodiment, the chewing gum base used for the composition of the present invention comprises resins. Suitable resins may include polyvinylacetate and terpene resins.

According to yet another embodiment, the chewing gum base used for the composition of the present invention comprises fats and/or oils, which may include including tallow, hydrogenated and partially hydrogenated vegetable oils, and cocoa butter.

According to yet another embodiment, the chewing gum base used for the composition of the present invention comprises waxes. Suitable waxes include paraffin, microcrystalline and natural waxes such as beeswax and carnauba.

According to yet another embodiment, the chewing gum base used for the composition of the present invention comprises softeners. Suitable softeners may include glycerol monostearate and glycerol triacetate.

According to yet another embodiment, the chewing gum base used for the composition of the present invention comprises inorganic fillers. Suitable filler components may be calcium carbonate, magnesium carbonate, talc, dicalcium phosphate or the like.

According to an expedient embodiment, the chewing gum base used for the composition of the present invention comprises 8-16% w/w of at least one elastomer, 12-19% w/w of polyvinyl acetate, 14-30% w/w of resin, 10-22% w/w of at least one hydrogenated and/or partially hydrogenated vegetable oil, 0-7% w/w of wax, 5-9% w/w of at least one emulsifier and/or technological assistant such as glycerol monostearate, acetylated monoglycerides, lecithin, sugar esters and/or triacetin, 15-40% w/w of inert mineral fillers, and 0-0.1% w/w of antioxidant.

In order to be applicable in the panning process, over 95% w/w of the particles of said free flowing powdered chewing gum composition must be below 1200 micron in diameter. To secure that the major part of the powdered chewing gum composition is deposited on the centres, and that the resulting coating is smooth and even, it is preferred that at least 65% w/w of the particles of the powdered chewing gum composition according to the invention have a particle size between 100 and 1000 micron. Even more preferably, at least 66% w/w, such as at least 67%, at least 68%, at least 69%, at least 70% w/w, at least 71%, at least 72%, at least 73% of the particles of the composition according to the invention should have a particle size between 100 and 1000 micron.

It is preferred that the powdered chewing gum composition according to the invention is a powder which is free flowing at temperatures between 5° C. and 30° C.

The powdered chewing gum composition may contain other ingredients well known in the art, such as flow-improving agents, antioxidants, colourants, flavours, bulking agents and sweeteners, e.g. bulk sweeteners, polyols or high intensity sweeteners.

Flow-improving agents may include talc, silica, silicate types such as magnesium and aluminumsilicate, titanium dioxide, magnesium stearate, gum arabic, and other suitable flow-improving agents and/or combinations thereof. Well known antioxidants include BHT, tocopherol, sodium erythorbate etc.

Bulk sweeteners are well known in the art and may include both carbohydrate sweeteners and carbohydrateless sweeteners, or combinations thereof. Carbohydrate sweeteners may include carbohydrate containing components including, but not limited to, sucrose, glucose, maltose, maltodextrin, dried invert sugar, dried agave syrup, dried grape juice, fructose, lactose, dried glucose syrups, and the like, alone or in combination. Carbohydrateless bulk sweeteners may include, but are not limited to polyols such as isomalt, sorbitol, mannitol, xylitol, hydrogenated starch hydrolysates, malititol and the like, alone or in combination. Further, carbohydrateless sweeteners include high intensity sweeteners, such as aspartame, saccharin and its salts, sucralose, stevioside, thaumatin, cyclamic acid and its salts, acesulfame and its salts, or any other suitable sweetener, alone or in combination.

Bulking agents may include carbohydrates or polyols which also provide sweetness to the product. However, since such materials are caloric, they may also be substituted with low-caloric bulking agents. Low caloric bulking agents may include inulin (Raftiline HP®, Beneo HPX®), Fructooligosaccarides (Raftilose® P95) (NutraFlora®), Isomaltulose (Palatinose®), Guar Gum Hydrosylate (Sun Fiber®), or indigestible dextrin (Fibersol-2® or Nutriose®), or any other suitable low caloric bulking agent.

The powdered chewing gum composition according to the present invention may contain a bulking agent and/or sweetening agent, said bulking and/or sweetening agent being used in an amount greater than about 1% w/w but not greater than 49%, preferably greater than about 10% w/w but not greater than 49%, more preferably greater than about 25% w/w but not greater than 49%, most preferred greater than about 35% w/w but not greater than 49%.

Colourants provide the desired colour characteristics or remove any undesired colour by whitening the gum portion of the coating. Powdered or liquid colouring agents may optionally be added during mixing of the powdered chewing gum composition. Colourants and whiteners may include, but are not limited to, fruit and vegetable extracts, titanium dioxide, rice starch or mixtures and/or dispersions thereof.

Flavouring agents may include essential oils, synthetic flavours or mixtures thereof including, but not limited to, flavouring aromatics, oleo resins, extracts derived from plants, leaves, flowers, fruits and so forth, and combinations thereof as known by those ordinarily skilled in the art. Flavour oils may include, but are not limited to, oils derived from plants and fruits, peppermint oil, spearmint oil, cinnamon oil, other mint oils, clove oil, oil of wintergreen, anise and the like. Also useful are artificial or natural fruit flavours such as citrus oils including lemon, orange, grape, lime and grapefruit and fruit essences including apple, strawberry, cherry, pineapple and so forth. Natural and artificial flavouring agents may be combined in any sensorial acceptable fashion.

The powdered chewing gum composition according to the present invention may contain a flavouring agent, said flavouring agent being used in an amount greater than about 0.1% w/w, preferably greater than about 2% w/w, more preferably greater than about 5% w/w. Thus, in a preferred embodiment the flavouring agent is used in an amount of between 0.1% w/w and 10% w/w, preferably between about 2% w/w, more preferably greater than about 5% w/w to about 10% w/w.

Particularly, in one aspect, the invention relates to a powdered chewing gum composition consisting essentially of:
a) 50% w/w to about 99% w/w chewing gum base,
b) about 1% w/w to about 45% w/w bulking and/or sweetening agent
c) about 0.1% w/w to about 10% w/w flavouring agent,
d) about 1% w/w to about 30% w/w talc, and optionally
e) about 0.1% w/w to about 20% w/w of a further flow improving agent

The present invention also relates to a chewing gum comprising a) a core compartment, and b) one or more layers comprising the composition according to the present invention.

In another aspect, the invention relates to a chewing gum comprising at least two distinct layers, wherein at least one layer contains a composition according to the invention.

In another aspect, the invention relates to a chewing gum comprising a) a core compartment, b) one or more intermediate layer compartments, and c) an outer layer compartment. Such chewing gums are commonly referred to as centre-filled chewing gums. According to this aspect of the invention, at least one of the intermediate layer compartments contains a composition according to the invention.

In the centre filled chewing gums according to the invention, the edible, chewable core compartment may comprise one or more compounds selected from confectioneries, nuts, dried or fresh fruits, flavourings, or preparations, such as tablets, capsules, sheets, compressed pills, wafers, easy melt tablets, semi-liquid or liquid preparations, the preparations comprising one or more active pharmaceutical ingredients (API's) and/or nutraceutical supplements, such as but not limited to, fish oil, omega-3 fatty acids, prebiotics, probiotic bacteria, caffeine, guarana, vitamins, minerals and enzymes.

The one or more intermediate compartments may comprise a solution acting as a glue (sugar containing or sugar free), and may further comprise flavour and colour, gum arabic, fine sugar or polyols for smoothening or a sealing component with a low permeability. However, at least one of the intermediate compartments must comprise a composition according to the invention.

The outer layer compartment preferably comprises no chewing gum base but is a finishing of the product for visual impact and/or a sealing of the product to reduce moisture absorption in order to improve shelf life. The outer layer may contain sugar and/or polyols, gum arabic, CMC, wax, fat, flavour and colour.

According to a preferred embodiment of the inventive chewing gum, the chewing gum composition constitutes at least 10% w/w, preferably at least 25% w/w, of the chewing gum according to the present invention. Hereby, it is ensured that the edible, chewable core compartment is surrounded by a quantity of chewing gum composition sufficient for sustained chewing of the chewing gum.

The present invention further comprises the use of a powdered chewing gum composition for coating a confectionery or a preparation, such as tablets, capsules, sheets, compressed pills, wafers, easy melt tablets, semi-liquid or liquid preparations, the preparation comprising one or more active pharmaceutical ingredients and/or nutraceutical supplements, such as but not limited to, fish oil, omega-3 fatty acids, prebiotics, probiotic bacteria, caffeine, guarana, vitamins, minerals and enzymes. The coating may be carried out in a standard panning apparatus such as a traditional coating pan or in a continuously operating pan. The process is well known in the industry and can be described as multiple layers on a centre. The layers are smoothened and compacted by the movement in the rotating pan by friction. First, a liquid solution of sugars/polyols and other ingredients and processing aids is added while the pan is rotating. When all the centres are wetted, the powdered chewing gum composition is added. The powder will stick to the wetted centres. When the centres are saturated with powder, they are wetted again and further powder material is added. This process is repeated until the desired weight is reached.

Accordingly, in another aspect, the invention relates to a method for production of a chewing gum according to the invention, the method comprising the steps of:
(a) providing a core composition; and
(b) coating of the core composition with a composition according to the present invention by use of a panning process; and optionally
(c) coating the outer intermediate layer with an outer finishing layer by use of a panning process.

### EXAMPLES

Any specifications herein are merely illustrative of the principles of the present invention, and are not to be seen as limiting in any way.

The aim of the experiments was to produce a piece of chewing gum using a powdered chewing gum composition via a standard coating process. The chewing gum should have a good chewability compared to standard chewing gum. A coating procedure was carried out in a 9-inch traditional stainless steel coating pan (standard panning apparatus).

In the coating is used the following coating materials and centres.

### Soft Coating Glue (Liquid):

1500 g maltitol syrup 75% d.s. (Maltisorb 75/75 Roquette Freres)
60 g gum arabic 396-I (Instant Senegal quality, Alland & Robert)
30 g food grade titanium dioxide
Heat under stirring to 70° C. and keep at this temperature.

### Pre-Gumming;

Pre-gumming is a coating step where cracks and un-even surfaces are smoothened for a better performance of the coating procedure and the over-all impression.

Pre-gumming is performed by adding a gum arabic solution (40% d.s. w/w) and dusting with Maltisorb P35 (maltitol crystalline in fine particles—average 35 micron).

The pre-gumming will also lower the migration of water from the centre (and into the centre) in-between the different coating materials.

### Hard Coating (Top Coating):

1040 g maltitol powder (Maltisorb P200, Roquette Freres)
15 g gum Arabic 396-I (Instant Senegal quality, Alland & Robert)
20 g food grade titanium dioxide
460 g water
Dissolve by stirring, and heat under stirring to 80° C.

### Powdered Chewing Gum Composition 1 (According to the Invention) Used in Example 1.

“GBP-T1”, a powdered chewing gum composition containing chewing gum base 58% (from Gum Base Company Italy), isomalt 12%, maltitol 10%, gum Arabic 10%, talc 10%. The product is produced according to inventor specifications.

### Powdered Chewing Gum Composition 2Gum (Standard Commercially Available) Used in Example 2.

“All in Gum”—Sugar Free (SF), Cafosa—a powdered chewing gum composition containing 24-26% chewing gum base and 74-76% sorbitol powder, flavour and colour free. Commercially available at Cafosa, Barcelona, Spain.

### Centres.

1500 gram soft compressed tablets, 10 mm diameter, weight 0.65 g each.

### Example 1

During 3.5 hours of active coating time 33 layers are applied according to table 1.

The resulting tablets have a 60/40 ratio between centre and coating.

The gum content is around 80% of the coating.

TABLE 1

| | | Amount | | Layer |
|---|---|---|---|---|
| Time (Minutes) | Product | (grams) | Comments | Number |
| 0:00 | Gum arabic solution | 25 | Pre gumming | 1. pre-layer |
| | 40% d.s. | | | |
| 0:01 | Maltisorb P35 | 100 | | |
| 0:05 | Gum arabic solution | 25 | | 2. pre-layer |
| | 40% d.s. | | | |
| 0:06 | Maltisorb P35 | 100 | | |
| 0:08 | Maltisorb P35 | 100 | | |
| 0:14 | Maltisorb P35 | 50 | Pause for 2 hours | |
| 0 (after pause) | Maltisorb 75/75 | 10 | Gum coating | 1. layer |
| 0:01 | GBP-T1 | 25 | | |
| 0:04 | Maltisorb 75/75 | 10 | | 2. layer |
| 0:06 | Maltisorb 75/75 | 10 | | |
| 0:07 | GBP-T1 | 25 | | |
| 0:12 | GBP-T1 | 25 | | |
| 0:15 | Maltisorb 75/75 | 10 | | 3. layer |
| 0:16 | GBP-T1 | 25 | | |
| 0:20 | Maltisorb 75/75 | 15 | | 4. layer |
| 0:21 | GBP-T1 | 25 | | |
| 0:25 | Maltisorb 75/75 | 15 | | 5. layer |
| 0:27 | GBP-T1 | 25 | | |
| 0:29 | GBP-T1 | 30 | | |
| 0:32 | Maltisorb 75/75 | 15 | | 6. layer |
| 0:34 | GBP-T1 | 30 | | |
| 0:36 | Maltisorb 75/75 | 15 | | 7. layer |
| 0:37 | GBP-T1 | 30 | | |
| 0:40 | GBP-T1 | 30 | | |
| 0:43 | Maltisorb 75/75 | 15 | | 8. layer |
| 0:44 | GBP-T1 | 30 | | |
| 0:47 | Maltisorb 75/75 | 15 | | 9. layer |
| 0:49 | GBP-T1 | 30 | | |
| 0:52 | Maltisorb 75/75 | 20 | | 10. layer |
| 0:53 | Menthol powder 80% | 10 | | |
| 0:54 | GBP-T1 | 40 | | |
| 1:00 | Maltisorb 75/75 | 20 | | 11. layer |
| 1:01 | GBP-T1 | 40 | | |
| 1:05 | Maltisorb 75/75 | 20 | | 12. layer |
| 1:08 | GBP-T1 | 40 | | |
| 1:12 | Maltisorb 75/75 | 20 | | 13. layer |
| 1:14 | GBP-T1 | 40 | | |
| 1:16 | Maltisorb 75/75 | 20 | | 14. layer |
| 1:17 | GBP-T1 | 40 | | |
| 1:19 | GBP-T1 | 40 | | |
| 1:22 | Maltisorb 75/75 | 20 | | 15. layer |
| 1:23 | GBP-T1 | 40 | | |
| 1:24 | GBP-T1 | 40 | | |
| 1:26 | Maltisorb 75/75 | 20 | | 16. layer |
| 1:27 | GBP-T1 | 40 | | |
| 1:29 | GBP-T1 | 40 | | |
| 1:31 | Maltisorb 75/75 | 20 | | 17. layer |
| 1:32 | GBP-T1 | 40 | | |
| 1:34 | GBP-T1 | 40 | | |
| 1:36 | Maltisorb 75/75 | 25 | | 18. layer |
| 1:37 | GBP-T1 | 40 | | |
| 1:38 | Peppermint flavour | 10 | | 19. layer |
| 1:39 | GBP-T1 | 40 | | |
| 1:40 | GBP-T1 | 40 | | |
| 1:41 | GBP-T1 | 40 | | |
| 1:47 | Maltisorb 75/75 | 25 | | 20. layer |
| 1:48 | GBP-T1 | 40 | | |
| 1:50 | GBP-T1 | 40 | | |
| 1:52 | Maltisorb 75/75 | 25 | | 21. layer |
| 1:53 | GBP-T1 | 40 | | |
| 1:55 | GBP-T1 | 40 | | |
| 1:57 | Maltisorb 75/75 | 25 | | 22. layer |
| 1:59 | GBP-T1 | 40 | | |
| 2:00 | GBP-T1 | 40 | | |
| 2:02 | Maltisorb 75/75 | 30 | | 23. layer |
| 2:04 | Splenda (sucralose) | 4 | | |
| 2:05 | GBP-T1 | 40 | | |
| 2:06 | GBP-T1 | 40 | | |
| 2:08 | Maltisorb 75/75 | 30 | | 24. layer |
| 2:09 | GBP-T1 | 40 | | |
| 2:10 | GBP-T1 | 40 | | |
| 2:12 | GBP-T1 | 40 | | |
| 2:14 | Maltisorb 75/75 | 30 | | 25. layer |
| 2:15 | GBP-T1 | 40 | | |
| 2:16 | GBP-T1 | 40 | | |
| 2:19 | Maltisorb 75/75 | 30 | | 26. layer |
| 2:20 | GBP-T1 | 40 | | |
| 2:21 | GBP-T1 | 40 | | |
| 2:24 | GBP-T1 | 40 | | |
| 2:26 | Maltisorb 75/75 | 30 | | 27. layer |
| 2:27 | Menthol powder 80% | 10 | | |
| 2:28 | GBP-T1 | 40 | | |
| 2:29 | GBP-T1 | 40 | | |
| 2:32 | Maltisorb 75/75 | 30 | | 28. layer |
| 2:33 | GBP-T1 | 40 | | |
| 2:34 | GBP-T1 | 40 | | |
| 2:37 | Peppermint flavour | 10 | | 29. layer |
| 2:38 | GBP-T1 | 40 | | |
| 2:39 | GBP-T1 | 40 | | |
| 2:40 | GBP-T1 | 40 | | |
| 2:41 | GBP-T1 | 40 | | |
| 2:43 | GBP-T1 | 40 | | |
| 2:46 | Maltisorb 75/75 | 30 | | 30. layer |
| 2:48 | GBP-T1 | 40 | | |
| 2:49 | GBP-T1 | 40 | | |
| 2:51 | Maltisorb 75/75 | 35 | | 31. layer |
| 2:53 | GBP-T1 | 40 | | |
| 2:54 | GBP-T1 | 40 | | |
| 2:58 | GBP-T1 | 40 | | |
| 2:59 | Maltisorb 75/75 | 35 | | 32. layer |
| 3:01 | GBP-T1 | 40 | | |
| 3.02 | GBP-T1 | 40 | | |
| 3:04 | GBP-T1 | 40 | | |
| 3:07 | Maltisorb P35 | 3 × 30 | Smoothening/drying | 33. layer |
| 3:25 | | | Finish gum coating | |
| | | | Hard coating | |
| Stage | 1 | 2 | 3 | 4 |
| (each of 5 min. | | | | |
| Addition | 25 ml. | 30 ml. | 35 ml. | 40 ml. |
| Number of | 5 | 15 | 30 | 5 |
| layers/repetition | | | | |
| Flavour stage | 10 ml. | 2 layers | peppermint oil | |
| Final stage | 35 ml. (brix 55) | 7 layers | smoothening | |

The chewing gum produced had a smooth surface, a fine texture and good chewability. The wet weight of the cud after chewing for 2 minutes was measured to 1.0 gr./piece. Flavour release is comparable with traditional chewing gum. The process was conducted without any problems during every phase of the trail.

### Example 2

During approx. 3.5 hours of active coating time, 33 layers are applied according to table 2.

TABLE 2

| | | Amount | | Layer |
|---|---|---|---|---|
| Time | Product | (grams) | Comments | Number |
| 0:00 | Gum arabic solution | 25 | Pre gumming | 1. pre- |
| | 40% d.s. | | | layer |
| 0:02 | Maltisorb P35 | 100 | | |
| 0:06 | Gum arabic solution | 25 | | 2. pre- |
| | 40% d.s. | | | layer |
| 0:07 | Maltisorb P35 | 100 | | |
| 0:08 | Maltisorb P35 | 100 | | |
| 0:15 | Maltisorb P35 | 50 | Pause for 2 hours | |
| 0:00 | Maltisorb 75/75 | 10 | Gum coating | 1. layer |
| 0:01 | All in Gum - SF | 25 | | |
| 0:06 | Maltisorb 75/75 | 10 | | 2. layer |
| 0:07 | All in Gum - SF | 40 | | |
| 0:12 | Maltisorb 75/75 | 10 | | 3. layer |
| 0:13 | All in Gum - SF | 40 | | |
| 0:18 | Maltisorb 75/75 | 15 | | 4. layer |
| 0:19 | All in Gum - SF | 40 | | |
| 0:24 | Maltisorb 75/75 | 15 | | 5. layer |
| 0:25 | All in Gum - SF | 40 | | |
| 0:29 | All in Gum - SF | 40 | | |
| 0:34 | Maltisorb 75/75 | 15 | | 7. layer |
| 0:35 | All in Gum - SF | 30 | | |
| 0:40 | All in Gum - SF | 30 | | |
| 0:45 | Maltisorb 75/75 | 15 | | 8. layer |
| 0:46 | All in Gum - SF | 40 | | |
| 0:49 | Maltisorb 75/75 | 15 | | 9. layer |
| 0:50 | All in Gum - SF | 45 | | |
| 0:54 | Sifting of “lumps” | | | |
| | balling up in the back | | | |
| | of the pan | | | |
| 0:54 | Maltisorb 75/75 | 20 | | 10. layer |
| 0:55 | Menthol powder 80% | 10 | | |
| 0:55 | All in Gum - SF | 45 | | |
| 1:00 | Maltisorb 75/75 | 20 | | 11. layer |
| 1:01 | All in Gum - SF | 45 | | |
| 1:05 | Maltisorb 75/75 | 20 | | 12. layer |
| 1:06 | All in Gum - SF | 45 | | |
| 1:11 | Maltisorb 75/75 | 20 | | 13. layer |
| 1:12 | All in Gum - SF | 45 | | |
| 1:17 | Maltisorb 75/75 | 20 | | 14. layer |
| 1:18 | All in Gum - SF | 45 | | |
| 1:23 | Maltisorb 75/75 | 20 | | 15. layer |
| 1:24 | All in Gum - SF | 45 | | |
| 1:25 | All in Gum - SF | 30 | | |
| 1:28 | Maltisorb 75/75 | 20 | | 16. layer |
| 1:29 | All in Gum - SF | 45 | | |
| 1:30 | All in Gum - SF | 30 | | |
| 1:34 | Sifting of “lumps” | | | |
| | balling up in the back | | | |
| | of the pan | | | |
| 1:34 | Maltisorb 75/75 | 20 | | 17. layer |
| 1:35 | All in Gum - SF | 45 | | |
| 1:36 | All in Gum - SF | 30 | | |
| 1:39 | Maltisorb 75/75 | 25 | | 18. layer |
| 1:40 | All in Gum - SF | 45 | | |
| 1:42 | Peppermint flavour | 10 | | 19. layer |
| 1:43 | All in Gum - SF | 45 | | |
| 1:48 | Maltisorb 75/75 | 25 | | 20. layer |
| 1:49 | All in Gum - SF | 45 | | |
| 1:53 | Maltisorb 75/75 | 25 | | 21. layer |
| 1:54 | All in Gum - SF | 45 | | |
| 1:55 | All in Gum - SF | 40 | | |
| 1:58 | Maltisorb 75/75 | 25 | | 22. layer |
| 1:59 | All in Gum - SF | 45 | | |
| 2:00 | All in Gum - SF | 45 | | |
| 2:03 | Sifting of “lumps” | | | |
| | balling up in the back | | | |
| | of the pan | | | |
| 2:03 | Maltisorb 75/75 | 30 | | 23. layer |
| 2:04 | Splenda (sucralose) | 3 | | |
| 2:05 | All in Gum - SF | 50 | | |
| 2:09 | Maltisorb 75/75 | 30 | | 24. layer |
| 2:10 | All in Gum - SF | 50 | | |
| 2:11 | All in Gum - SF | 40 | | |
| 2:15 | Maltisorb 75/75 | 30 | | 25. layer |
| 2:16 | All in Gum - SF | 50 | | |
| 2:16 | All in Gum - SF | 40 | | |
| 2:20 | Maltisorb 75/75 | 30 | | 26. layer |
| 2:21 | All in Gum - SF | 50 | | |
| 2:22 | All in Gum - SF | 40 | | |
| 2:27 | Maltisorb 75/75 | 30 | | 27. layer |
| 2:28 | Menthol powder 80% | 10 | | |
| 2:28 | All in Gum - SF | 50 | | |
| 2:29 | All in Gum - SF | 40 | | |
| 2:33 | Sifting of “lumps” | | | |
| | balling up in the back | | | |
| | of the pan | | | |
| 2:33 | Maltisorb 75/75 | 30 | | 28. layer |
| 2:34 | All in Gum - SF | 50 | | |
| 2:35 | All in Gum - SF | 40 | | |
| 2:39 | Peppermint flavour | 10 | | 29. layer |
| 2:40 | All in Gum - SF | 50 | | |
| 2:45 | All in Gum - SF | 50 | | |
| 2:49 | All in Gum - SF | 40 | | |
| 2:59 | Sifting of “lumps” | | | |
| | balling up in the back | | | |
| | of the pan | | | |
| 3:00 | Maltisorb 75/75 | 35 | | 32. layer |
| 3:01 | All in Gum - SF | 50 | | |
| 3.02 | All in Gum - SF | 50 | | |
| 3:05 | All in Gum - SF | 40 | | |
| 3:12 | Maltisorb P35 | 3 × 30 | Smoothening/drying | 33. layer |
| 3:20 | | | Finish gum coating | |
| 3:20 | Sifting of “lumps” | | | |
| | balling up in the back | | | |
| | of the pan | | | |
| | | | Hard coating | |

The chewing gum produced was evaluated as not usable, as the powdered chewing gum composition was washed off from the centres during the coating process and ended up in the siftings/lumps (the smaller balls in the back of the pan). The cud-size of the chewing gum was for this reason close to zero and the product can only be characterised as a coated tablet.

1-14. (canceled)

15. A chewing gum comprising a) a core compartment, and b) one or more layers comprising a powdered chewing gum composition, characterised in that the composition contains over 50% w/w chewing gum base, and further characterised in that at least 95% w/w of the particles of the composition have a particle size of less than 1200 micron in diameter.

16. The chewing gum according to claim 15, characterised in that the composition contains between 50% w/w and 90% w/w chewing gum base.

17. The chewing gum according to claim 16, characterised in that the composition contains between 70 and 85% w/w chewing gum base.

18. The chewing gum according to claim 15, wherein the chewing gum base has a softening point of between 50-65° C.

19. The chewing gum according to claim 18, wherein the chewing gum base has a softening point of 58-62° C.

20. The chewing gum according to claim 15, wherein at least 65% w/w of the particles of the composition have a particle size of between 100 and 1000 micron.

21. The chewing gum according to claim 15, wherein said composition is a powder which is free flowing at temperatures between 5° C. and 30° C.

22. The chewing gum according to claim 15, wherein said composition further comprises one or more of sweeteners, bulking agents, flavouring agents, colourants, antioxidants, and flow-improving agents.

23. The chewing gum according to claim 15, wherein the composition essentially consists of:
a) 50% w/w to about 99% w/w chewing gum base,
b) about 1% w/w to about 45% w/w bulking and/or sweetening agent
c) about 0.1% w/w to about 10% w/w flavouring agent,
d) about 1% w/w to about 30% w/w talc, and optionally
e) about 0.1% w/w to about 20% w/w of another flow improving agent.

24. The chewing gum according to claim 15, further comprising c) an outer layer compartment.

25. The chewing gum according to claim 15, wherein the core compartment comprises a compound selected from confectioneries, nuts, dried or fresh fruits, flavourings, one or more active pharmaceutical or nutraceutical ingredients.

26. The chewing gum according to claim 15, wherein the composition constitutes at least 10% w/w of the chewing gum.

27. A method for production of a chewing gum comprising the steps of:
(a) providing a core composition; and
(b) coating of the core composition with a powdered chewing gum composition, characterised in that the powdered chewing gum composition contains over 50% w/w chewing gum base, and further characterised in that at least 95% w/w of the particles of the powdered chewing gum composition have a particle size of less than 1200 micron in diameter, by use of a panning process; and optionally
(c) coating the outer intermediate layer with an outer finishing layer by use of a panning process.

28. The chewing gum according to claim 25, wherein the one or more active pharmaceutical or nutraceutical ingredients is selected from fish oil, omega-3 fatty acids, prebiotics, probiotic bacteria, caffeine, guarana, vitamins, minerals and enzymes.
